(19) United States
(12) Patent Application Publication
(10) Pub. No.: US 2011/0313148 A1
(43) Pub. Date: Dec. 22, 2011

(54) AUTOMATED OLIGOSACCHARIDE SYNTHESIZER

(76) Inventors: William Christ, Andover, MA (US); Lenz Krock, Zurich (CH); Obadiah J. Plante, Danvers, MA (US); Bastien Castagner, Zurich (CH); Peter H. Seeberger, Kleinmachnow (DE)

(21) Appl. No.: 13/055,401

(22) PCT Filed: Jul. 23, 2009
(86) PCT No.: PCT/US09/51517

§ 371 Date: Sep. 8, 2011

Related U.S. Application Data
(60) Provisional application No. 61/135,722, filed on Jul. 23, 2008.

(51) Int. Cl. C07H 3/06; C07H 1/06; B01J 19/00; C07H 1/00

(52) U.S. Cl. 536/123.1; 422/105

(57) ABSTRACT

The technical field of this invention is automated oligosaccharide synthesizers. There is a need in this field for more efficient oligosaccharide synthesizers. For example, the present invention is an apparatus for solid phase oligosaccharide synthesis, which includes a reaction vessel for holding a reaction mixture, such that the reaction vessel is equipped with a temperature control system, a donor vessel for holding a saccharide donor; an activation vessel for holding activator, a pump operably connected to a fluidic valve; an additional fluidic valve connected to the activation vessel, to the first fluidic valve via a first fluid line, and to the reaction vessel via a second fluid line, such that the activator or saccharide donor can be delivered via the second fluidic valve into the first fluid line and then through the second fluid line into the reaction vessel.

### CROSS-REFERENCES TO RELATED APPLICATIONS

The present patent document claims the benefit of the filing date under 35 U.S.C. §119(e) of Provisional U.S. Patent Application Ser. No. 61/135,722, filed Jul. 23, 2008, which is hereby incorporated by reference.

### BACKGROUND

The present invention is an automated oligosaccharide synthesizer.

Biopolymers, such as polypeptides and polynucleotides, are routinely synthesized by solid-phase methods in which polymer subunits are added stepwise to a growing polymer chain immobilized on a solid support. For polynucleotides and polypeptides, this general synthetic procedure can be carried out with commercially available synthesizers that construct the biopolymers with defined sequences in an automated or semi-automated fashion. However, commercially available synthesizers do not allow the efficient synthesis of oligosaccharides; typically, the yields and quality of oligosaccharides synthesized using the commercially available apparatus are poor.

The glycosylation reaction is one of the most thoroughly studied transformations in organic chemistry. In the most general sense, a glycosylation is the formation of an acetal connecting two sugar units. The majority of glycosylating agents follow similar paths of reactivity. The anomeric substituent acts as a leaving group thereby generating an electrophilic intermediate or transition state. Reaction of this species with a nucleophile, typically a hydroxyl group, leads to the formation of a glycosidic linkage. This reaction may proceed via a number of intermediates depending on the nature of the leaving group, the activating reagent and the solvent employed.

Glycosyl trichloroacetimidates, thioglycosides, N-phenyl trifluoroacetimidates, glycosyl sulfoxides, glycosyl halides, glycosyl phosphites, n-pentenyl glycosides and 1,2-anhydrosugars are among the most reliable glycosyl donors. Despite the wealth of glycosylating agents available, no single method has been distinguished as a universal donor. Contrary to peptide and oligonucleotide synthesis, the inherent differences in monosaccharide structures make it unlikely that a common donor will prevail. Rather, individual donors will see use in the construction of certain classes of glycosidic linkages.

Solution-phase oligosaccharide synthesis remains a slow process due to the need for iterative coupling and deprotection steps with purification at each step along the way. To alleviate the need for repetitive purification events, solid-phase techniques have been developed. In solid-phase oligosaccharide synthesis there are two methods available. The first, the donor-bound method, links the first sugar to the polymer through the non-reducing end of the monomer unit. The polymer-bound sugar is then converted into a glycosyl donor and treated with an excess of acceptor and activator. Productive couplings lead to polymer bound disaccharide formation while decomposition products remain bound to the solid support. Elongation of the oligosaccharide chain is accomplished by converting the newly added sugar unit into a glycosyl donor and reiteration of the above cycle. Since most donor species are highly reactive, there is a greater chance of forming polymer-bound side-products using the donor-bound method.

In a second method, the acceptor bound method, the first sugar is attached to the polymer at the reducing end. Removal of a unique protecting group on the sugar affords a polymer-bound acceptor. The reactive glycosylating agent is delivered in solution and productive coupling leads to polymer-bound oligosaccharides while unwanted side-products caused by donor decomposition are washed away. Removal of a unique protecting group on the polymer-bound oligosaccharide reveals another hydroxyl group for elongation.

While the merits of the donor-bound method have been demonstrated by Danishefsky and co-workers, the most popular and generally applicable method of synthesizing oligosaccharides on a polymer support remains the acceptor-bound strategy. For a review, see: P. H. Seeberger, S. J. Danishefsky, Acc. Chem. Res., 31 (1998), 685. The ability to use excess glycosylating agents in solution to drive reactions to completion has led to widespread use of this method. All of the above mentioned glycosylating agents have been utilized with the acceptor-bound method to varying degrees of success.

U.S. Pat. No. 7,160,517 describes an automated oligosaccharide synthesizer. The present invention provides an improved system.

### BRIEF SUMMARY

In one aspect, the present invention provides an apparatus for solid phase oligosaccharide synthesis, comprising a reaction vessel for holding a reaction mixture, wherein the reaction vessel is equipped with a temperature control system, at least one donor vessel for holding a saccharide donor; at least one activation vessel for holding activator, a pump operably connected to a first fluidic valve; a second fluidic valve connected to the activation vessel, to the first fluidic valve via a first fluid line, and to the reaction vessel via a second fluid line, wherein activator or saccharide donor can be delivered via the second fluidic valve into the first fluid line and then through the second fluid line into the reaction vessel.

In another aspect, the present invention provides an apparatus for solid phase oligosaccharide synthesis, comprising a reaction vessel for holding a reaction mixture, with a temperature control system for controlling the temperature within the reaction vessel, at least one deblocking vessel for holding a deblocking reagent; at least one donor vessels for holding a saccharide donor; and at least one activation vessel for holding activator; a solution transfer system connecting the activation vessel, deblocking vessel, and donor vessel to the reaction vessel; and a computer for controlling the temperature control system and the solution transfer system; wherein the computer system is programmed to regulate the addition of activator into the reaction vessel based on the temperature within the reaction vessel.

In other aspects, the above apparatus can further comprise additional fluidic valves operably connected to additional vessels and fluid lines, such that the contents of the additional vessels can be isolated from the saccharide donor and activator and from other fluid lines but can still be delivered to the reaction vessel via the same (or an additional) pump.

In the above apparatus, each fluidic valve can be a rotary valve, solenoid valve block or other multi-port valve or valve system. In the above apparatus, each pump can be a syringe pump, a peristaltic pump or other suitable pump.

In another aspect, the present invention provides a method comprising adding a glycosyl acceptor immobilized on a solid support to a reaction vessel of an automated synthesizer; wherein the automated synthesizer comprises the reaction vessel; a pump operably connected to a first fluidic valve; a second fluidic valve operably connected to a donor vessel holding saccharide donor, to the first fluidic valve via a first fluid line, to a reaction vessel via a second fluid line, and, optionally to an activator vessel holding activator, adding saccharide donor via the second fluidic valve into the first fluid line and then through the second fluid line into the reaction vessel; and adding activator into the reaction vessel to form a product immobilized on the solid support.

In one aspect, the present invention provides a method comprising adding a glycosyl acceptor immobilized on a solid support to a reaction vessel of an automated synthesizer; wherein the temperature within the reaction vessel is monitored by a temperature control system, a computer and a heating and/or cooling unit surrounding the reaction vessel; adding a glycosyl donor to the reaction vessel, adding an amount of activator to the reaction vessel to form a mixture at a reaction temperature; monitoring the temperature of the mixture and adjusting the temperature of the reaction vessel so as to substantially maintain the temperature of the mixture within ±1° C. of the reaction temperature, and repeating steps (c) through (d) at least one more time to form a product which is the glycosyl donor bonded to the glycosyl acceptor via a saccharide bond, wherein there is a period of time between step (a) and (e) where no activator is added to the reaction vessel.

The above methods can further comprise a washing step, a deblocking step, further coupling and deblocking steps, and/or a decoupling from the solid support step.

### BRIEF DESCRIPTION OF THE DRAWINGS

FIG. 1 is an illustration of an automated synthesizer in accordance with the present invention, where the solution transfer system includes a single syringe pump.

FIG. 2 is a schematic of the fluidic valves (V1-6) shown in FIG. 1.

FIG. 3 is an illustration of another embodiment of the automated synthesizer in accordance with the present invention, where the solution transfer system includes two syringe pumps.

FIG. 4 is a schematic of the fluidic valves shown in FIG. 3.

FIG. 5A is a drawing of the top of the reaction vessel illustrated in FIG. 1;

FIG. 5B is a side view of the reaction vessel top.

FIG. 6 is an illustration of a heating/cooling unit used with a reaction vessel with a sealed bottom.

### DETAILED DESCRIPTION OF THE DRAWINGS AND THE PRESENTLY PREFERRED EMBODIMENTS

In this application, the following nomenclature is used: V# refers to a specific fluidic valve (i.e., V4 is fluidic valve 4); V#P# refers to a specific port position on a specific fluidic valve (i.e., V2P1 refers to fluidic valve 2, port position 1); L# refers to a specific loop (i.e., L2 is loop 2).

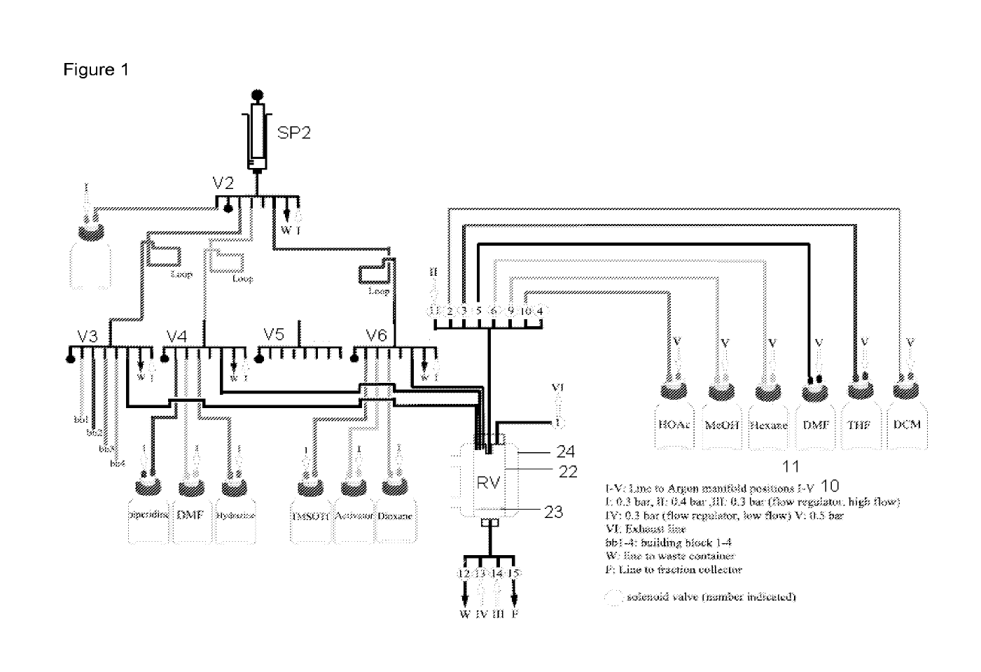

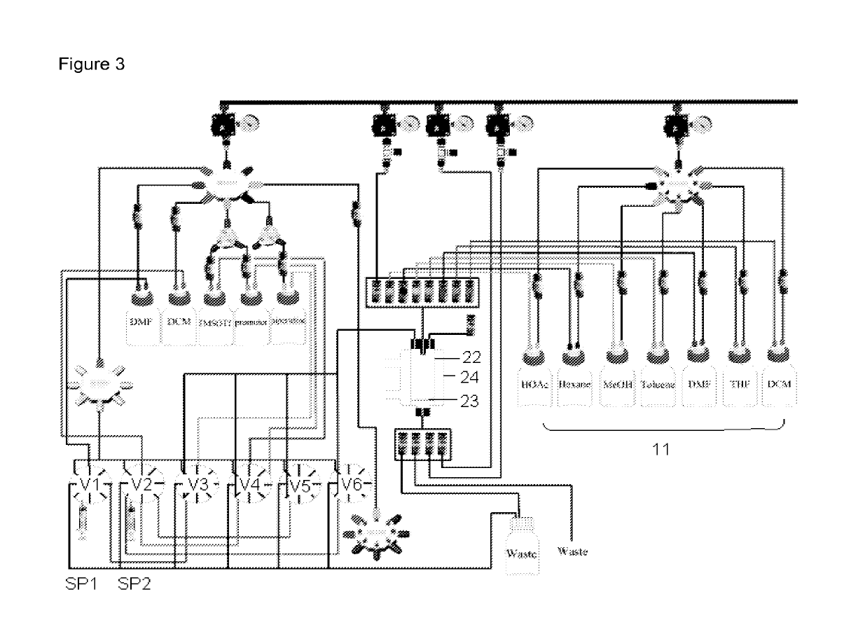

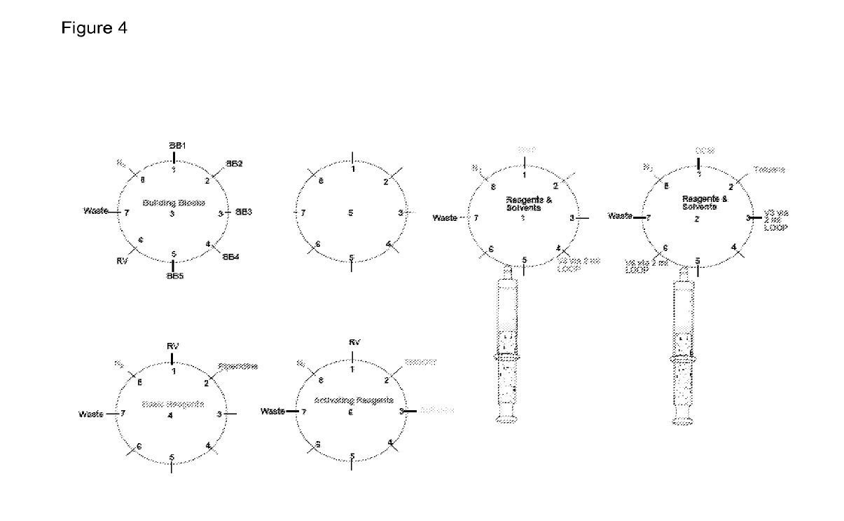

In FIG. 1, a device with a solution transfer system with a single pump (SP2) is used. In FIG. 3, a device using solution transfer system with two pumps (SP1 and SP2) is illustrated. Any pump can be used in accordance with the present invention, including syringe pumps, peristaltic pumps and others known to those skilled in the art.

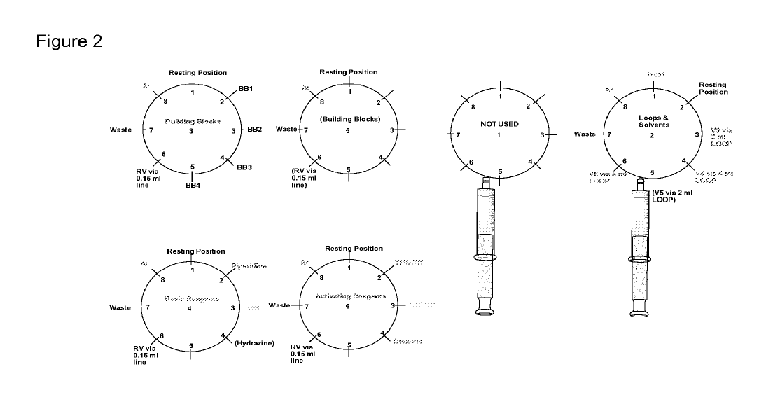

In FIG. 1, SP2 is connected to V2. FIG. 2 details the port configurations for V1. In FIGS. 1 and 2, the fluidic valve is shown as a rotary valve with 8 ports. It should be understood that FIGS. 1 and 2 detail the configuration of one apparatus in accordance with the present invention. Other configurations are possible, so long as they are based on the guiding principles set forth below (e.g., see FIG. 3). Suitable fluidic valves include rotary valves (such as those available from J-KEM Scientific, Inc. (St. Louis, Mo.) or Kloehn Ltd. (Las Vegas, Nev.)), or solenoid valve blocks (such as those available from OmniFit or J-KEM).

In FIG. 2, SP1 and V1 are not used in synthesis, but are instead available for back up use. SP2 is connected to V2 which has eight ports. V1P1 is connected to solvent (DCM shown); V1P2 (the resting position) is preferably connected to a bottle or, alternatively, is plugged; V1P3, V1P4, V1P5 and V1P6 are connected to individual loops; V1P7 is connected to waste; and V1P8 is connected to an inert gas (Argon shown). One aspect of the invention is that the pumps are not directly connected to reagent. Instead, only solvent or inert gas is directly connected to a pump (e.g., solvent or inert gas is drawn into the syringe of a syringe pump.

SP2 is indirectly connected to reagent via the loops attached at V1P3, V1P4, V1P5 and V1P6. Each loop is thus connected to V1 (or V2 if in use) in addition to one other fluidic valve. Regents can be grouped by reactivity. As shown in FIGS. 2, V3 and V4 are associated with building block reagents; V4 is associated with basic or deblocking reagents; and V6 is associated with activating reagents. As each fluidic valve is associated with only one loop, reagents of similar reactivity can be isolated from those with different reactivity, preventing cross-contamination. Further since reagent is drawn into a loop instead of into the pump, the pump is subject to less wear and reduced risk of cross-contamination of reagents.

The loops are ideally constructed from an inert material such as, for example, Teflon, poly(tetrafluoroethylene) (PTFE), polypropene (PPE), etc.

The size of the loops can be varied. The exact size will depend on the capacity of the syringe pump (defining the maximum size) and the amount of reagent to be delivered to the reaction vessel (defining the minimum size). The size of each loop will also depend on the nature of the reagent to which it is associated. For example, if the reaction vessel is 20 mL, then a loop sized from about 1 to 5 mL may be used; preferably from about 2 to 4 mL. Each loop can be sized the same or different. For example, loops attached to building blocks may be smaller than those attached to basic reagents as the quantity of the former used during any synthetic step is relatively small compared to the amount of basic reagent.

In FIG. 2, both V3 and V5 have the same port configuration. That is, V3P1 and V5P1 are the resting position. As noted above for V2, the resting position port can either be connected to a bottle or alternatively plugged (e.g., with a Teflon plug). The resting positions ideally are chosen to match the default settings applied when the system is started. Under normal conditions, upon start the SP2 is emptied. If the syringe is empty, a plugged resting port is suitable. However, if the syringe is full (e.g., when the system restarts after a power failure in mid-synthesis), a plugged resting port could result in destruction of the port or the syringe. To avoid this, the resting positions preferably are connected to a bottle, such that the syringe can empty into the bottle.

V3P2-5 and V5P2-5 can be connected to individual building blocks. In FIG. 2, four building blocks are in use: V3P2-5 are connected to building blocks (BB) 1-4 respectively; while V5P2-5 are not in use. If V5P2-5 were in use, eight building blocks could be used in the synthesis. In an alternate embodiment, some or all of these port positions could connect to additional fluidic valves with similar port configurations via loops (enabling the use of more than 8 building blocks in the synthesis).

V3P6 and V5P6 are connected to the reaction vessel 22. V3P7 and V5P7 are connected to waste. V3P8 and V5P8 are connected to an inert gas (argon shown).

In FIG. 2, the basic and activating reagents are distributed respectively on V4 and V6, respectively. As with the other fluidic valves, V4P1 and V6P1 are the resting position. V4P2-5 can be connected to up to four basic reagents, or alternatively as explained above can be connected via loops to further fluidic valves similarly configured to increase the number of basic reagents used. In FIG. 2, only two reagents are illustrated: V4P2 is connected to piperidine and V4P4 is connected to hydrazine. For V6, V6P2-5 can be connected to up to four activating reagents, or alternatively as explained above can be connected via loops to further fluidic valves similarly configured to increase the number of activating reagents used. In FIG. 2, V6P2 is connected to TMSOTf and V6P4 is connected to dioxane. As with V3 and V5, V4P6 and V6P6 are connected to the reaction vessel; V4P7 and V6P7 are connected to waste; V4P8 and V6P8 are connected to an inert gas.

Returning to FIG. 1, solvents 11 are separated from the reaction vessel 22 by a solenoid valve block 12. Solvents are ideally kept blanketed and/or pressurized with an inert gas 10. When a solenoid valve is opened, the corresponding solvent flows into the reaction vessel. When the same solenoid valve is closed, no solvent flows.

In FIG. 1, reagents are also blanketed and/or pressurized with an inert gas 10. The gas line used to pressurize the reagents can be the same or different from that used with the solvents. Whereas solvent flow into the reaction vessel 22 is controlled by the solenoid valve block, reagent flow into the reaction vessel 22 is controlled by the fluidic valves and pump described above. The system is blanketed to prevent oxygen degradation of the solvents and reagents and to prevent moisture from entering the system. The system is preferably pressurized to allow reagents and solvent to be added quickly.

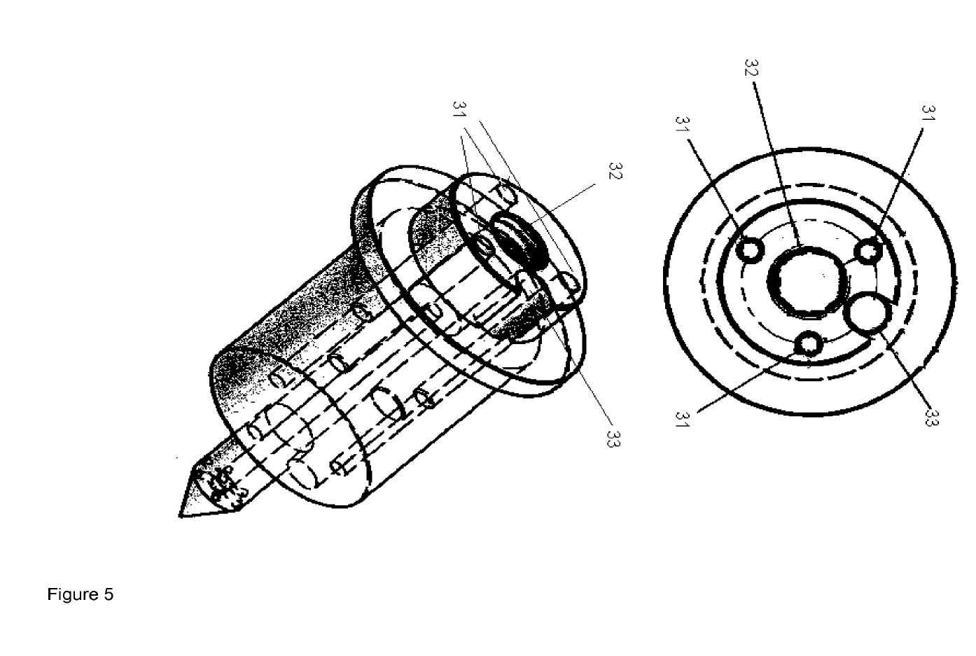

The reaction vessel 22 in FIG. 1 is fitted with a top. The top is shown in more detail in FIGS. 5A and B. The top is configured to receive reagent or solvent from V3, V4, V5 or V6 (holes 31); to receive solvent via the solenoid block (hole 32); and to vent gas via exhaust line VI (hole 33). When the reaction vessel is sealed on the bottom, the top must have an additional opening for an outlet line. When the reaction vessel is open on the bottom (such as depicted in FIGS. 1 and 3), the bottom of the reaction vessel is fitted with a frit 23. Flow out of the reaction vessel is controlled by solenoid valves 12-15. The frit is sized to retain the solid support in the reaction vessel 22.

In either case (seal or unsealed at bottom), the chamber of the reaction vessel is sized to accommodate the solid support, reagents and solvent. Typically, the chamber holds between 1 mL and 100 mL of solvent, more preferably 5-20 mL.

The reaction vessel in FIG. 1 is surrounded by a temperature control unit 24. The temperature control unit 24 can be any suitable device which capable of regulating and maintain the temperature of the reaction vessel 22 at a desired temperature(s). Typically, the reaction vessel 22 is maintained at a temperature of between about −80° C. and +60° C., and preferably between about −25° C. and +40° C. It is contemplated that the temperature control system should be able to maintain the temperature within the reaction vessel and, if necessary, adjust the temperature to within ±1° C. of the reaction temperature. For example, by monitoring the temperature within the reaction vessel (versus the bath), the temperature can be adjusted to account for exotherms caused by the reaction.

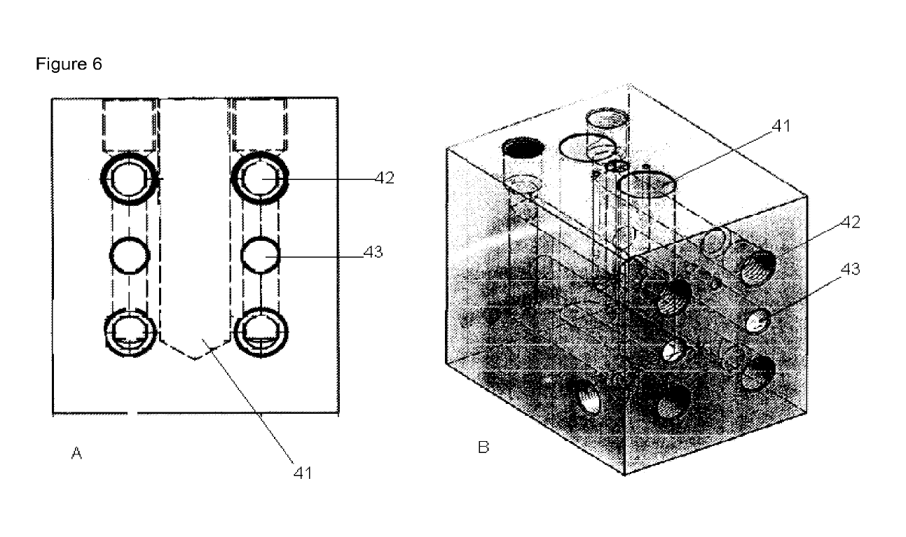

In one embodiment, the temperature control unit 24 can be as simple as a heating and/or cooling unit equipped with a thermometer, where the unit temperature can be adjusted either manually or by a computer. For example, the unit could be a heating bath, an external refrigerated circulator such as those available from the Julabo USA, Inc. (Allentown, Pa.), a heating/cooling block such as shown in FIG. 6.

In FIG. 6, the heating/cooling block can be made of any heat transfer material such as aluminum. The block has channels 42 running through to pass coolant through as well as channels 43 for heating elements. The reaction vessel sits in channel 41. When a heating/cooling block such as shown in FIG. 6 is used, the reaction vessel is sealed at the base. In this embodiment, the reaction vessel 22 not only has to have inlet lines 31 from V3P6, V4P6, V6P6, but also an outlet line (not shown) (controlled by a pump that can be the same or different than the pump in the solution transfer system). To prevent the solid support from being drawn into the outlet line, the end in the reaction vessel is fitted with a frit or filter (not shown). To evacuate the reaction vessel after a reaction step or washing step, a vacuum is pulled on the outlet line. Such vacuum can be produced by withdrawal of the plunger in syringe pump SP2.

In another embodiment, the system allows more sophisticated control. Coolant can be circulated around the reaction vessel 22 via a sleeve surrounding the reaction vessel 22 and connected to the temperature control unit 24 via input and output pathways. Alternatively, the reaction vessel 22 can be a double-walled structure wherein the external cavity of the double-walled structure accommodates the coolant of the temperature control unit 24. The temperature of the reaction vessel 22 can be established by pre-programming the temperature control unit 24 to a desired, fixed temperature and then allowing the coolant to circulate around the reaction vessel 22. Alternatively, the temperature control unit 24 can have a temperature sensor placed on the wall of the reaction vessel 22 or, preferably, in the reaction vessel 22, so as to obtain real-time temperature measurements of the actual reaction vessel 22 cavity, i.e., where the synthesis of the oligosaccharides are to take place. Thus, the temperature sensor can provide feedback data to the temperature control unit 24 so that the actual temperature of the reaction vessel 22 can more properly be maintained.

The temperature control unit 24 can also be linked to the operation of the pumps and fluidic valves. That is, during coupling reactions, rather than adding reagent (e.g., activator) in one aliquot to the reaction vessel, it instead can be metered into the reaction vessel based on the temperature inside the reaction vessel 22. In this manner, temperature spikes that may impact the stereochemistry of the forming glycosidic bond or undesirable side-reactions can be avoided. The synthesizer of the present invention is especially designed with this feature in mind. By first pulling reagents into loops, versus delivering them directly to the reaction vessel, one can control the addition of specific reagents into the reaction vessel.

The pumps, fluidic valves and temperature control unit are preferably computer controlled.

The Model 433A peptide synthesizer available from the Applied Biosystems Inc. (CA) can be modified to obtain an automated synthesizer in accordance with the present invention. Some modifications have been previously described in U.S. Pat. No. 7,160,517. Other modifications are shown in FIGS. 1 and 2. In particular, the ABI solution transfer system and the system described in U.S. Pat. No. 7,160,577 are both assemblies of zero dead volume valves in a valve block. Reagent is in a tube with an attached liquid sensor. Reagent is passed from the tube into the valve block with a calibrated flow resistance and at a fixed known pressure, so that the length of time required for a transfer corresponds directly to the volume of material which is transferred. The reagent then is passed from the valve block into the reaction vessel in a single injection.

The inventive solution transfer system profoundly differs from the above described prior art systems. Whereas those systems added an amount of activator into the reaction vessel in a single injection, the inventive system allows the addition of the activator into the reaction vessel as the coupling is progressing, either continuously or through periodic introduction of sub-stoichiometric amounts. The inventive system contemplates the flow of activator into the reaction vessel based on the rate of reaction. As coupling reaction proceeds (as monitored via temperature), additional amounts of activator can be added until the reaction is complete. For example, activator could be added into the reaction vessel if the reaction vessel temperature is within ±1° C. of the desired reaction temperature but halted if this value is exceeded. In this way, the stereoselectively, cleanliness and yield of the coupling can be increased compared to the stereoselectivity obtained when activator is added as a single injection. By controlling the addition of activator into the reaction vessel, the stereoselectivity of the resulting product can be improved. Ideally, the stereoselectivity of each formed glycosidic bound is greater than 50%, preferably greater than about 75%, more preferably greater than about 95%, and most preferably greater than 99%.

Method of Use

The automated synthesizer of the present invention is intended to be used to form oligo- and polysaccharides on solid support via repeated coupling and deblocking steps.

Suitable solid supports are well known in the art and include octenediol functionalized 1% crosslinked polystyrene, SynPhase Lanterns™, etc.

Suitable building blocks are well known in the art and include glycosyl trichloroacetimidate donors, thioglycoside donors, etc.

Suitable protecting groups for the building blocks are well known in the art. For example, chapter 3 of Lindhorst, “Essentials of Carbohydrate Chemistry and Biochemistry” 2^{nd} ed., WILEY-VCH Verlag GmbH & Co. (Weinheim Del.), 2003, is dedicated to a discussion of suitable protecting groups for carbohydrates, including acyl, ether, acetal, orthoester, etc. Preferred protecting groups include ester and silyl groups.

Suitable activators are well known in the art and include trimethylsilyl trifluoromethanesulfonate (TMSOTf), BF₃ etherate, trifluoromethanesulfonic acid (TfOH), Pd(CH₃CN)₄BF₄, etc.

Suitable deblocking agents (basic reagents) are well known in the art and include piperidine, hydrazine, sodium methoxide in methanol, 1 M butylamine in tetrahydrofuran (THF), etc.

Coupling Cycles

In a typical coupling cycle, the glycosyl donor and the activator are delivered to the solid support and allowed to react. After a suitable time (typically 1 hour), the solid support is rinsed and the coupling repeated to maximize coupling. Thereafter, the solid support is rinsed and washed several times to produce glycosyl-bound solid support. Then, in a typical deblocking step, a basic reagent is introduced in the reaction vessel and allowed to react with the glycosyl bound-solid support. After a suitable time (typically 30 min), the solid support is rinsed.

Deletion sequences (those missing just one or more sugar unit(s) (n−1)) are the most difficult to separate from the desired product and arise from incomplete coupling steps during any coupling cycle of the sequence. The oligosaccharide chains that fail to couple during one cycle, may be successfully glycosylated during the following elongation steps. Therefore, a severe purification problem may exist at the end of the synthesis. To avoid the elongation of failure sequences, a capping step (i.e., a blocking step) can be included into the coupling cycle. After each completed coupling, a highly reactive blocking group can be used to cap any free hydroxyl acceptors. For example, benzyl trichloroacetimidate can be employed as a capping reagent (activated with TMSOTf) to yield benzyl ethers in positions that were not glycosylated and render them unreactive throughout the synthesis. Also, fluorous capping agents could be used such as those described by Seeberger (Angew. Chem. Int. Ed. 2001, 40, 4433). Using this straightforward capping step, the purification of the finished oligosaccharide products is expected to be greatly simplified, since the presence of deletion sequences will be minimized.

If further sugars are to be added, the coupling and deblocking steps are repeated.

Following the completion of the synthesis, the polysaccharide is removed from the solid support.

Polysaccharide can be purified and characterized using methods well known in the art.

The invention now being generally described, it will be more readily understood by reference to the following examples, which are included merely for purposes of illustration of certain aspects and embodiments of the present invention, and are not intended to limit the invention.

### EXAMPLE

### General Synthetic Scheme

A reaction vessel is loaded with solid support (e.g., octenediol functionalized solid support) and inserted into the oligosaccharide synthesizer. A temperature control unit is set to maintain the temperature in the chamber of the reaction vessel at 25° C. Solenoid valves 12-15 are closed and solenoid valves 11 and 1 are open (and remain open throughout synthesis) in FIG. 1.

Glycosylation of the solid support is carried out by treating the solid support with a building block (e.g., glycosyl donor in DCM) and slowly metering in activator (e.g., TMSOTf in DCM). The solid support is then washed several times with solvent (e.g., DCM—6×4 mL each) and glycosylated a second time with building block/activator. Upon completion of the double glycosylation, the solid support is washed with solvents (e.g., DCM—6×4 mL each, followed by a mixture of MeOH/DCM—4×4 mL each).

Referring to FIG. 1, the flow of regent for the glycosylation step is as follows: Donor (bbl) is drawn into a loop between V3 and SP2 (the fluidic valves are positioned at V2P3, V3P2, V4P1, V6P1). Donor is then delivered to the reaction vessel (the fluidic valves are positioned at V2P3, V3P6, V4P1, V6P1). Activator is then drawn into a loop between V6 and SP2 (the fluidic valves are positioned at V2P6, V3P1, V4P1, V6P2). Under control of the temperature control unit, activator is periodically delivered to the reaction vessel (the fluidic valves are positioned at V2P6, V3P1, V4P1 or V6P6 (depending on reaction temperature), V6P1). The loop can be washed with solvent by drawing solvent into the syringe pump (the fluidic valves are positioned at V2P1, V3P1, V4P1, V6P1), with the solvent delivery through the loop into the waste (the fluidic valves are positioned at V2P3, V3P7, V4P1, V6P1) or into the reaction vessel (the fluidic valves are positioned at V2P3, V3P6, V4P1, V6P1).

After all the activator is delivered and the reaction is complete the fluidic valves are closed (the fluidic valves are positioned at V2P2, V3P1, V4P1, V6P1) and remaining reagent is removed from the reaction vessel via the solenoid valves (12 opens). The beads in the reaction vessels can be washed with a solvent 11 by opening one of solenoid valves 2, 3, 5, 6, 9 or 10. After the beads are washed, all of the solenoid valves close (except 11 and 1).

Deprotection of the acetyl ester is carried out by treating the glycosylated solid support with a basic reagent (e.g., piperidine). The solid support is then washed with solvent (e.g., a mixture of MeOH/DCM (1×4 mL) and subjected to the deprotection conditions a second time. Removal of any soluble impurities is accomplished by washing the solid support with solvent (e.g., a mixture of MeOH/DCM—4×4 mL each; then 0.2 M AcOH in THF—4×4 mL each; then THF—4×4 mL each; and finally DCM—6×4 mL each).

Referring to FIG. 1, the flow of reagent for the deprotection step is as follows: Basic reagent (piperidine) is drawn into a loop between V4 and SP2 (the fluidic valves are positioned at V2P4, V3P1, V4P2, V6P1). Basic reagent is then delivered to the reaction vessel (the fluidic valves are positioned at V2P4, V3P1, V4P6, V6P1). Additional basic reagent can be added by repeating the sequence. The loop can be washed with solvent by drawing solvent into the syringe pump (the fluidic valves are positioned at V2P1, V3P1, V4P1, V6P1), with the solvent delivery through the loop into the waste (the fluidic valves are positioned at V2P4, V3P1, V4P7, V6P1) or into the reaction vessel (the fluidic valves are positioned at V2P4, V3P1, V4P6, V6P1).

The deprotected polymer bound acceptor is then elongated by reiteration of the above glycosylation/deprotection protocol, using different building blocks, activators, deprotecting agents, and solvents as determined by the operator and programmed into the solution transfer system.

1. An apparatus for solid phase oligosaccharide synthesis, comprising:
a reaction vessel for holding a reaction mixture, wherein the reaction vessel is equipped with a temperature control system,
at least one donor vessel for holding a saccharide donor;
at least one activation vessel for holding activator,
a pump operably connected to a first fluidic valve;
a second fluidic valve connected to the activation vessel, to the first fluidic valve via a first fluid line, and to the reaction vessel via a second fluid line,
wherein activator or saccharide donor can be delivered via the second fluidic valve into the first fluid line and then through the second fluid line into the reaction vessel.

2. The apparatus of claim 1, further comprising:
a third fluidic valve operably connected to the donor vessel, to the first fluidic valve via a third fluid line, and to the reaction vessel via a fourth fluid line;
wherein saccharide donor can be delivered via the third fluidic valve into the third fluid line and then through the fourth fluid line into the reaction vessel and wherein activator can be delivered via the second fluidic valve into the first fluid line and then through the second fluid line into the reaction vessel.

3. The apparatus of claim 1, further comprising a deblocking vessel for holding a basic reagent, wherein the basic reagent can be delivered via the second fluidic valve into the first fluid line and then through the second fluid line into the reaction vessel.

4. The apparatus of claim 3, further comprising a deblocking vessel for holding a basic reagent, wherein the basic reagent can be delivered via the third fluidic valve into the third fluid line and then through the fourth fluid line into the reaction vessel.

5. The apparatus of claim 2, further comprising
a deblocking vessel for holding a basic reagent,
a fourth fluidic valve operably connected to the deblocking vessel, to the first fluidic valve via a fifth fluid line, and to the reaction vessel via a sixth fluid line;
wherein basic reagent can be delivered via the fourth fluidic valve into the fifth fluid line and then through the sixth fluid line into the reaction vessel.

6. The apparatus of claim 1, wherein each fluidic valve is a rotary valve.

7. The apparatus of claim 1, wherein the pump is syringe pump.

8. A method comprising
(a) adding a glycosyl acceptor immobilized on a solid support to a reaction vessel of an automated synthesizer; wherein the automated synthesizer comprises:
(1) the reaction vessel;
(2) a pump operably connected to a first fluidic valve;
(3) a second fluidic valve operably connected to a donor vessel holding saccharide donor, to the first fluidic valve via a first fluid line, to a reaction vessel via a second fluid line, and, optionally to an activator vessel holding activator,
(b) adding saccharide donor via the second fluidic valve into the first fluid line and then through the second fluid line into the reaction vessel; and
(c) adding activator into the reaction vessel to form a product immobilized on the solid support.

9. The method of claim 8, wherein the apparatus further comprises a third fluidic valve operably connected to the first fluidic valve via a third fluid line, to the reaction vessel via a fourth fluid line, and to an activator vessel holding activator;
wherein step (c) comprises adding activator via the third fluidic valve into the third fluid line and then through the fourth fluid line into the reaction vessel to form a product immobilized on the solid support.

10. The method of claim 8, further comprising (d) washing the solid support and then repeating steps (b), (c) and (d) at least one more time.

11. The method of claim 8, further comprising:
(e) deblocking the product of step (d);
(f) washing the solid support; and then
(g) repeating steps (a) to (f) at least 2 more times so as to form an oligosaccharide immobilized on the solid support.

12. The method of claim 11, further comprising the step of (h) decoupling the oligosaccharide from the solid support.

13. An apparatus for solid phase oligosaccharide synthesis, comprising:
a reaction vessel for holding a reaction mixture,
with a temperature control system for controlling the temperature within the reaction vessel,
at least one deblocking vessel for holding a deblocking reagent;
at least one donor vessel for holding a saccharide donor; and
at least one activation vessel for holding activator;
a solution transfer system connecting the activation vessel, deblocking vessel, and donor vessel to the reaction vessel; and
a computer for controlling the temperature control system and the solution transfer system; wherein the computer system is programmed to regulate the addition of activator into the reaction vessel based on the temperature within the reaction vessel.

14. A method comprising
(a) adding a glycosyl acceptor immobilized on a solid support to a reaction vessel of an automated synthesizer; wherein the temperature within the reaction vessel is monitored by a temperature control system, a computer and a heating and/or cooling unit surrounding the reaction vessel;
(b) adding a glycosyl donor to the reaction vessel,
(c) adding an amount of activator to the reaction vessel to form a mixture at a reaction temperature;
(d) monitoring the temperature of the mixture and adjusting the temperature of the reaction vessel so as to substantially maintain the temperature of the mixture within ±1° C. of the reaction temperature, and
(e) repeating steps (c) through (d) at least one more time to form a product which is the glycosyl donor bonded to the glycosyl acceptor via a saccharide bond, wherein there is a period of time between step (a) and (e) where no activator is added to the reaction vessel.

15. The method of claim 14, further comprising:
(f) deblocking the product of step (e);
(g) repeating steps (a) to (f) at least 2 more times so as to form an oligosaccharide.

16. The method of claim 15, further comprising the step of (h) decoupling the oligosaccharide from the solid support.

17. The method of claim 14, wherein the total amount of activator used in the method is less than or equal to the stiochiometric amount of glycosyl donor.
